(19) United States
(12) Patent Application Publication
(10) Pub. No.: US 2019/0076471 A1
(43) Pub. Date: Mar. 14, 2019

(54) DIETARY SUPPLEMENTS, ADDITIVES, AND DRENCH FOR LIVESTOCK

(71) Applicant: John D. Robinson, Cylinder, IA (US)

(72) Inventor: John D. Robinson, Cylinder, IA (US)

(21) Appl. No.: 16/131,645

(22) Filed: Sep. 14, 2018

Related U.S. Application Data
(60) Provisional application No. 62/558,636, filed on Sep. 14, 2017.

(51) Int. Cl. A61K 33/10; A61K 33/42; A61K 33/14; A61K 33/00; A61K 33/04; A61K 33/06; A61K 33/32; A61K 33/30; A61K 33/26; A61K 33/34; A61K 31/07; A61K 31/355; A61K 31/122; A61K 36/062; A61K 31/593; A61K 31/455; A61K 31/525; A61K 31/714; A61K 31/51; A61K 35/747; A61K 35/744; A61K 9/00; A23K 20/22; A23K 20/24; A23K 20/26; A23K 20/20; A23K 10/18; A23K 50/10

(52) U.S. Cl. CPC A61K 33/10; A61K 2035/115; A61K 33/14; A61K 33/00; A61K 33/04; A61K 33/06; A61K 33/32; A61K 33/30; A61K 33/26; A61K 33/34; A61K 31/07; A61K 31/355; A61K 31/122; A61K 36/062; A61K 31/593; A61K 31/455; A61K 31/525; A61K 31/714; A61K 31/51; A61K 35/747; A61K 35/744; A61K 9/0056; A23K 20/22; A23K 20/24; A23K 20/26; A23K 20/30; A23K 10/18; A23K 50/10; A61K 33/42

(57) ABSTRACT

A first livestock feed additive comprises calcium, phosphorus, sodium chloride, potassium, sulfur, magnesium, manganese, zinc, iron, copper, selenium, vitamin A, Vitamin E, and mendione; a second livestock feed additive comprising sodium bentonite, sodium sulfate, dried Aspergillus oryzae fermentation product, ferrous sulfate, calcium carbonate, manganese proteinate, manganese sulfate, molasses products, vegetable oil, lecithin, high fructose corn syrup, polysorbate 60, glycerin, sulfuric acid, xanthan gum, natural and artificial flavor, zinc proteinate, selenium yeast, zinc sulfate, yeast culture, vitamin A supplement, vitamin D-3 supplement, vitamin E supplement, choline chloride, niacin, calcium pantothenate, riboflavin, vitamin B-12 supplement, thiamine hydrochloride, copper sulfate, copper proteinate, Lactobacillus acidophilus, Lactobacillus brevis, Lactobacillus plantarum, Enterococcus faecium, and Pediococcus acidlacticii; and a livestock drench composition comprising crude protein, crude fat, fiber, copper, manganese, zinc, vitamin E, phosphorus, sodium chloride, magnesium, potassium, iron, iodine, and sulfur.

### CROSS-REFERENCE TO RELATED APPLICATIONS

This application claims priority under 35 U.S.C. § 119 to provisional application U.S. Ser. No. 62/558,636, filed Sep. 14, 2017. The provisional patent application is herein incorporated by reference in its entirety, including without limitation, the specification, claims, and abstract, as well as any figures, tables, appendices, or drawings thereof.

### FIELD OF THE INVENTION

The present invention relates to improved heath and performance of livestock produced by novel compositions and methods involving formulations provided herein. The present invention may be applied to a variety of livestock, including ruminants such as cattle and sheep.

### BACKGROUND OF THE INVENTION

The background description provided herein is for the purpose of generally presenting the context of the present disclosure. Work of the presently named inventors, to the extent the work is described in the present disclosure, as well as aspects of the description that may not otherwise qualify as prior art at the time of filing, are neither expressly nor impliedly admitted as prior art.

The conventional dietetic approach to feedlotting of ruminants is to feed high proportions of grain (>70%) and low proportions of roughage (<20%) in the diet. The aim of this strategy is to maximize the digestibility of the grain and its constituent nutrients including starch and proteins in the rumen. However, there is considerable variation in the starch content of different grains. For example, wheat contains about 70-75% starch, corn and sorghum 68-72% starch, and oats and barley 56-60% starch. Further, there are large differences in the rumen digestibility of starch for the different grains. For example, the starch sources present in wheat and barley are highly digested in the rumen (84-90%) whereas that of sorghum is much lower (60-65%). Such variation in starch content and rumen digestibility results in major differences in feedlot performance and in turn this has significant implications for profitability.

When assessing the nutrient requirements of animals in feedlots it is also necessary to take into account their stage of physiological growth and maturity. For example, at the beginning of beef feedlotting cattle invariably undergo an adaptation phase which involves transition from free range grazing to intensive feedlotting, and commensurately, there is a major change in nutrient supply from a forage to a grain based diet. In effect, this creates an enormous metabolic challenge for the animal and invariably results in compensatory phases of growth occurring. This generally occurs in the first 40-50 days of feedlotting and during this period there is an additional demand for essential amino acids, essential fatty acids and glucose to satisfy intestinal growth and development, which increases during this compensatory/adaptation phase. A further example where different supplies of nutrient are required is in fat deposition and in particular the accumulation of intramuscular fat—a process commonly referred to as marbling.

Efficient rumen fermentation in ruminants is important to maximizing animal performance, including feed intake, feed efficiency, production of milk, meat, or other animal products, and maintaining acceptable levels of milk components. A healthy rumen environment reduces the risk and incidence of metabolic disorders that can occur with improper feeding management or abrupt feed intake changes, such as acidosis.

Current cattle industry practices incorporate the genetic selection of certain characteristics such as feed efficiency or maximized milk production. Genetic selection can result in animals that gain weight efficiently and/or produce large volumes of high quality milk. Genetically selected cattle are fed energy dense rations to maximize milk production. Energy dense rations can inhibit fiber fermentation and increase the difficulty of maintaining an optimal rumen environment. The feeding of high concentrate diets for cattle has increased awareness of problems associated with ruminal acidosis and the implications it has on feed consumption, feed efficiency, production of milk, meat, or other animal products, and animal health.

Current practices also include self-feeding for certain animals, which may include finishing beef and finishing dairy beef cattle. Self-fed nutrition management programs provide all or part of an animal's complete diet in feeder equipment that allows free access for the animal to this diet at all times of the day over the entire feeding period. Self-feeding often occurs in dry lot environments, but can occur in other environments as well. Typically, self-fed programs are designed for feeding beef and dairy cattle to the appropriate weight and fatness level whereby the resulting meat satisfies the standards of the meat industry. Self-fed programs can also be provided at any time during the growing and development periods for animals to achieve their desired weight and maturity. Because animals lack the nutritional wisdom to regulate their feed consumption to optimize health, growth, and beneficial rumen microbial growth, acute and subacute digestive disorders can result in these self-fed environments.

A substantial drop in ruminal pH can damage the rumen wall, decrease blood pH, cause dehydration, laminitis, or erratic feed consumption patterns, and severely depress feed intake and feed efficiency. During acidosis and the recovery period, females may also experience decreased milk production.

Thus, there is a need in the art to provide compositions and methods for improving livestock health and performance, to maintain a healthy rumen pH, and to prevent disorders such as acidosis.

### SUMMARY OF THE INVENTION

Therefore, it is a primary object, feature, or advantage of the present invention to improve on or overcome the deficiencies in the art.

It is still yet a further object, feature, or advantage of the present invention to provide compositions and methods which produce improvements in feed intake, feed efficiency, productive milk output, milk quality indices, and/or meat production.

It is still yet a further object, feature, or advantage of the present invention to provide a feed supplement which provides improved availability of at least one nutrient or an improvement in the energy gained from a feed material.

It is still yet a further object, feature, or advantage of the present invention provide a drench composition which provides improved availability of at least one nutrient or an improvement in the energy gained from a feed material during times when the animal is under stress.

It is still yet a further object, feature, or advantage of the present invention to provide dietary supplements that are safe for consumption.

It is still yet a further object, feature, or advantage of the present invention to provide dietary supplements that are cost effective.

It is still yet a further object, feature, or advantage of the present invention to provide dietary supplements that are stable and have a long shelf life.

It is still yet a further object, feature, or advantage of the present invention to provide dietary supplements that are easily manufactured, stored, and transported.

It is still yet a further object, feature, or advantage of the present invention to provide livestock whose health are improved by the consumption of dietary supplements accomplishing some or all of the previously stated objectives.

It is still yet a further object, feature, or advantage of the present invention to provide methods of manufacturing and consuming dietary supplements accomplishing some or all of the previously stated objectives.

According to some aspects of the present disclosure, a livestock feed additive comprises calcium, phosphorus, sodium chloride, potassium, sulfur, magnesium, manganese, zinc, iron, copper, selenium, vitamin A, Vitamin E, and mendione.

According to some other aspects of the present disclosure, the livestock feed additive comprises sodium bentonite, sodium sulfate, dried Aspergillus oryzae fermentation product, ferrous sulfate, calcium carbonate, manganese proteinate, manganese sulfate, molasses products, vegetable oil, lecithin, high fructose corn syrup, polysorbate 60, glycerin, sulfuric acid, xanthan gum, natural and artificial flavor, zinc proteinate, selenium yeast, zinc sulfate, yeast culture, vitamin A supplement, vitamin D-3 supplement, vitamin E supplement, choline chloride, niacin, calcium pantothenate, riboflavin, vitamin B-12 supplement, thiamine hydrochloride, copper sulfate, copper proteinate, Lactobacillus acidophilus, Lactobacillus brevis, Lactobacillus plantarum, Enterococcus faecium, and Pediococcus acidlacticii.

According to some other aspects of the present disclosure, a livestock drench composition comprises crude protein, crude fat, fiber, copper, manganese, zinc, vitamin E, phosphorus, sodium chloride, magnesium, potassium, iron, iodine, and sulfur.

The novel compositions of the present disclosure are intended to be added to a conventional livestock feed, although they can be given alone or in combination with other additives. The supplement and drench each can be added to the feed is in the form of a powder, liquid, or solid. The supplement and drench each may also be provided as, or integrated into, a feed block. The compositions may be otherwise administered orally. The oral administration of enhanced feed in the form of a liquid or suspension also presents numerous additional benefits to the bovine producers. For example, benefits are seen as a result of increased ease of application and use, decreased cost and improved convenience of administration.

In another aspect of the present disclosure, the compositions of the present invention can be used for improving the ruminal health of a ruminant animal. Improvements in ruminal health through use of the compositions can include, for example, increased nutrient availability and absorption by the animal, increased fermentation, and increased rumen microbial growth.

There are also numerous additional benefits to the quality of the meat derived from cattle provided with the compositions of the present invention. These benefits are derived in addition to the significant benefits seen in increasing the health and performance of the livestock.

These or other objects, features, and advantages of the present invention will be apparent to those skilled in the art after reviewing the following detailed description of the illustrated embodiments, accompanied by the attached drawings.

### DETAILED DESCRIPTION OF THE INVENTION

The following definitions and introductory matters are provided to facilitate an understanding of the present invention. Unless defined otherwise, all technical and scientific terms used herein have the same meaning as commonly understood by one of ordinary skill in the art to which embodiments of the present invention pertain.

The terms “a,” “an,” and “the” include plural referents unless context clearly indicates otherwise. Similarly, the word “or” is intended to include “and” unless context clearly indicate otherwise. The word “or” means any one member of a particular list and also includes any combination of members of that list.

The terms “invention” or “present invention” as used herein are not intended to refer to any single embodiment of the particular invention but encompass all possible embodiments as described in the specification and the claims.

The term “about” as used herein refers to variation in the numerical quantities that can occur, for example, through typical measuring techniques and equipment, with respect to any quantifiable variable, including, but not limited to, mass, volume, time, distance, wave length, frequency, voltage, current, and electromagnetic field. Further, given solid and liquid handling procedures used in the real world, there is certain inadvertent error and variation that is likely through differences in the manufacture, source, or purity of the ingredients used to make the compositions or carry out the methods and the like. The claims include equivalents to the quantities whether or not modified by the term “about.”

The apparatuses, systems, and methods of the present invention may comprise, consist essentially of, or consist of the components of the present invention described herein. The term “consisting essentially of” means that the apparatuses, systems, and methods may include additional components or steps, but only if the additional components or steps do not materially alter the basic and novel characteristics of the claimed apparatuses, systems, and methods.

The following embodiments are described in sufficient detail to enable those skilled in the art to practice the invention however other embodiments may be utilized. Mechanical, procedural, and other changes may be made without departing from the spirit and scope of the invention. Accordingly, the scope of the invention is defined only by the appended claims, along with the full scope of equivalents to which such claims are entitled.

The present invention relates to improved health and performance of livestock through provision of compositions, including compositions provided as a food supplement or additive. Applicant provides a special enrichment diet for livestock such as beef cattle, dairy cattle, horses, swine, poultry, goats, sheep, and the like, providing numerous benefits, including, for example, improved muscle mass and tone, fertility, hair growth, eye health, feed intake, feed efficiency, productive milk output, milk quality indices, meat production, and/or reduced morbidity and mortality.

In a preferred embodiment of the methods of the present invention for feeding livestock, the health and performance of livestock can be enhanced by feeding at least one composition in an amount of, for example, at least 0.125 oz. per head, 0.25 oz per head, 0.5 oz per head, 1 oz. per head, or 2 oz. per head. The compositions may be provided for a limited period of time, or may be provided over the lifetime of the animal. In a further embodiment, the feed additive and the drench compositions may be feed separately or in combination with one another. A further preferred feeding range consists of feeding livestock throughout its life with the special enriched diet. The dietary composition can be further enriched with or provided in addition to organic trace minerals and vitamins.

In a further embodiment, the feed additive comprises calcium, phosphorus, sodium chloride, potassium, sulfur, magnesium, manganese, zinc, iron, copper, selenium, vitamin A, Vitamin E, and mendione. In a further embodiment, the feed additive comprises sodium bentonite, sodium sulfate, dried Aspergillus oryzae fermentation product, ferrous sulfate, calcium carbonate, manganese proteinate, manganese sulfate, molasses products, vegetable oil, lecithin, high fructose corn syrup, polysorbate 60, glycerin, sulfuric acid, xanthan gum, natural and artificial flavor, zinc proteinate, selenium yeast, zinc sulfate, yeast culture, vitamin A supplement, vitamin D-3 supplement, vitamin E supplement, choline chloride, niacin, calcium pantothenate, riboflavin, vitamin B-12 supplement, thiamine hydrochloride, copper sulfate, copper proteinate, Lactobacillus acidophilus, Lactobacillus brevis, Lactobacillus plantarum, Enterococcus faecium, and Pediococcus acidlacticii.

In a further embodiment, the drench composition comprises crude protein, crude fat, fiber, copper, manganese, zinc, vitamin E, phosphorus, sodium chloride, magnesium, potassium, iron, iodine, and sulfur.

In a still further embodiment, the feed additive and/or drench compositions may include probiotic bacteria such as, Lactobacillus acidophilus, Lactobacillus brevis, Lactobacillus plantarum, Enterococcus faecium, and Pediococcus acidlacticii. Additional yeast species, vitamins, and minerals may also be included dependent on the animal and its health.

The dietary supplements or additives according to the present invention can be formulated substantially according to Tables 1 and 2, below.

TABLE 1
Exemplary nutrient solution of a feed additive

| | First | Second | Third |
|---|---|---|---|
| Component | Embodiment | Embodiment | Embodiment |
| Calcium | 1-10% | 2-8% | 2-6% |
| Phosphorus | 0.1-1% | 0.25-1% | 0.5-0.75% |
| Sodium | 0.1-1% | 0.5-1% | 0.6-0.8% |
| chloride | | | |
| Potassium | 0.01-0.2% | 0.1-0.2% | 0.1-0.15% |
| Sulfur | 0.01-0.25% | 0.08-0.25% | 0.08-0.1% |
| Magnesium | 0.01-0.1% | 0.025-0.075% | 0.04-0.06% |
| Manganese | 0.1-2% | 0.25-1.5% | 0.5-1.5% |
| Zinc | 0.05-0.5% | 0.1-0.5% | 0.1-0.25% |
| Iron | 0.1-1% | 0.2-0.6% | 0.4-0.55% |
| Copper | 0-0.1% | 0.0-0.05% | 0.001-0.01% |
| Selenium | 0-0.01% | 0.0001-0.005% | 0.0001-0.001% |
| Vitamin A | 20-75 KIU/lb | 30-60 KIU/lb | 35-50 KIU/lb |
| Vitamin E | 200-500 IU/lb | 300-450 IU/lb | 400-420 IU/lb |
| Menadione | 25-75 mg/lb | 45-60 mg/lb | 50-55 mg/lb |

TABLE 2
Exemplary nutrient solution of a drench composition

| Component | First Embodiment | Second Embodiment | Third Embodiment |
|---|---|---|---|
| Crude protein | 0.05-2% | 1-2% | 1-1.5% |
| Crude fat | 0.001-0.1% | 0.001-0.1% | 0.01-0.03% |
| Crude fiber | 0.0001-0.01% | 0.001-0.01% | 0.001-0.003% |
| Copper | 30-90 ppm | 50-90 ppm | 50-70 ppm |
| Manganese | 50-125 ppm | 75-125 ppm | 75-105 ppm |
| Zinc | 50-125 ppm | 75-125 ppm | 75-105 ppm |
| Vitamin E | 50-180 IU/lb | 75-180 IU/lb | 115-165 IU/lb |
| Phosphorus | 0.01-1% | 0.25-1% | 0.25-0.5% |
| Sodium | 0.01-1% | 0.15-1% | 0.15-0.4% |
| Magnesium | 0.01-1% | 0.1-1% | 0.1-0.3% |
| Potassium | 0.01-1% | 0.05-1% | 0.05-0.3% |
| Iron | 0.0001-0.1% | 0.008-1% | 0.008-0.01% |
| Iodine | 1-25% | 12-25% | 12-17% |
| Sulfur | 0-15% | 0-10% | 0-5% |

TABLE 3
Exemplary component solution of a feed additive

| | First | Second | Third |
|---|---|---|---|
| Component | Embodiment | Embodiment | Embodiment |
| Sodium Bentonite | 20-60% | 35-60% | 35-45% |
| Sodium Sulfate | 10-50% | 25-50% | 25-30% |
| Dried Aspergillus | 0-20% | 5-20% | 5-10% |
| oryzae Fermentation | | | |
| Product | | | |
| Ferrous Sulfate | 0-20% | 5-20% | 5-10% |
| Calcium Carbonate | 0-20% | 5-20% | 5-10% |
| Manganese Proteinate | 0-10% | 1-10% | 1-5% |
| Manganese Sulfate | 0-10% | 1-10% | 1-5% |
| Molasses/Vegetable | 0-10% | 1-10% | 1-5% |
| Oil Blend | | | |
| Zinc Proteinate | 0-10% | 1-10% | 1-5% |
| Selenium Yeast | 0-10% | 0-10% | 1-5% |
| Zinc Sulfate | 0-5% | 0.5-5% | 0.5-2% |
| Yeast Culture | 0-5% | 0.5-5% | 0.5-2% |
| RAF Beef Blue (B- | 0-5% | 0.5-5% | 0.5-2% |
| Vitamins) | | | |
| Copper Sulfate | 0-5% | 0.5-2% | 0.5-2% |
| Copper Proteinate | 0-5% | 0.5-2% | 0.5-2% |
| Vitamin E 50% | 0-1% | 0.01-1% | 0.01-0.5% |
| Life 10-G Microbial | 0-1% | 0.01-1% | 0.01-0.5% |

The results of feeding the special enhanced diet of the present invention to livestock illustrate that animals exhibiting stress or inability to thrive (i.e., foundering offspring) exhibit substantial improvements in health and performance following administration of dietary supplements according to the present invention. The improvements include increases in muscle tissue, hair growth, fertility, feed intake, feed efficiency, productive milk output, milk quality indices, and/or meat production. There is evidence suggesting the dietary supplements can be used both to improve the performance and health of livestock not exhibiting any disease or defect, and to treat livestock exhibiting defects and/or disease symptoms, including, for example, poor growth or failure to thrive.

Further, the formulations according to the present invention can also be used for livestock other than cattle. However, it should be understood that the formulae may require modification for administration for certain livestock. For example, removal of copper is required before administration of sheep.

The invention has been described with reference to various specific and preferred embodiments and techniques. However, it should be understood that many variations and modifications may be made while remaining within the spirit and scope of the invention. In particular, it is to be understood that the present invention contemplates variations in the organic components of the special enriched diet, process parameters for making the special enriched diet, including temperature, time, pH, salts, manufacturing techniques, drying or preparation techniques, and selenoproteins of interest.

All publications in this specification are indicative of the level of ordinary skill in the art to which this invention pertains. All publications are herein incorporated by reference in its entirety to the same extent as if each individual publication was specifically and individually indicated by reference.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

Sixteen male goats were purchased and initially weighed. Blood samples were taken from each goat as well to establish a baseline health and confirm no abnormalities existed in the animals. Each pen contained four goats of relatively equal starting weight. Pens were then fed for 26 days as described in Table 3. Materials feed are as follows:
Hubbard Feed: 57% corn; 19% oats; 21% Hubbard Life Good Supplement for Meat & Dairy Goats (Available from Hubbard Feed); 0.4% molasses; 2.6% soy oil. Formulated for 16% crude protein.
Big Gain Feed: #9252RAA Complete Goat Grower XQP RU, Available from Big Gain, Inc. Formulated for 16% crude protein.

TABLE 4
Feeding Programs for Example 1

| | |
|---|---|
| Pen 1 | Hubbard Feed with feed additive according to disclosure |
| Pen 2 | Hubbard Feed (control) |
| Pen 3 | Big Gain Feed with feed additive according to disclosure |
| Pen 4 | Big Gain Feed (control) |

After 26 days of feeding, weights and blood samples were taken from each goat. Baseline weights and blood test results are shown in Table 5. Results after 26 days of feeding according to Table 4 are shown in Table 6.

TABLE 5
Baseline Weights and Blood Results
19 Jul. 2017

| Goat | Tag | | | | | | Total | Albu- | | Creatine | Alk | | Total | Hemolytic | Lipemic | Iceteric |
|---|---|---|---|---|---|---|---|---|---|---|---|---|---|---|---|---|
| No. | ID | Pen | Wt. | BUN | Creat | Glucose | Protein | min | AST | Kinase | Phos | GGT | Bilirubin | Indice | Indice | Indice |
| 1 | 26 | 1 | 67 | 13 | 0.9 | 52 | 6.1 | 2.5 | 79 | 101 | 93 | 93 | 0.36 | 15 | 20 | 2 |
| 2 | 27 | 1 | 65 | 14 | 0.9 | 47 | 6.3 | 2.5 | 81 | 85 | 139 | 98 | 0.55 | 43 | 20 | 2 |
| 3 | 28 | 1 | 62 | 5 | 0.7 | 62 | 5.5 | 2.2 | 94 | 88 | 110 | 178 | 0.42 | 46 | 20 | 2 |
| 4 | 29 | 1 | 62 | 10 | 0.8 | 60 | 6.9 | 2.9 | 135 | 176 | 511 | 127 | 0.69 | 50 | 20 | 2 |
| | | Avg. | 64 | 10.5 | 0.83 | 55.25 | 6.2 | 2.53 | 97.25 | 112.5 | 213.25 | 124 | 0.51 | 38.5 | 20 | 2 |
| 7 | 32 | 2 | 51 | 9 | 0.6 | 52 | 6 | 2.4 | 107 | 135 | 138 | 90 | 0.43 | 25 | 20 | 2 |
| 8 | 33 | 2 | 52 | 6 | 0.7 | 48 | 6.6 | 2.9 | 120 | 280 | 92 | 107 | 0.93 | 124 | 20 | 2 |
| 9 | 34 | 2 | 53 | 13 | 0.7 | 64 | 5.7 | 2.2 | 90 | 81 | 202 | 9 | 0.55 | 20 | 20 | 2 |
| 15 | 40 | 2 | 48 | 11 | 0.7 | 51 | 7 | 2.9 | 107 | 158 | 272 | 108 | 0.62 | 43 | 20 | 2 |
| | | Avg. | 51 | 9.75 | 0.68 | 53.75 | 6.325 | 2.6 | 106 | 163.5 | 176 | 78.5 | 0.63 | 53 | 20 | 2 |
| 5 | 30 | 3 | 65 | 13 | 0.6 | 49 | 7 | 3.1 | 115 | 93 | 198 | 128 | 0.71 | 79 | 20 | 2 |
| 13 | 38 | 3 | 57 | 9 | 0.6 | 48 | 6.8 | 2.8 | 92 | 59 | 172 | 121 | 0.52 | 15 | 20 | 2 |
| 14 | 39 | 3 | 65 | 4 | 0.7 | 67 | 6.3 | 2.9 | 103 | 72 | 256 | 123 | 0.45 | 41 | 20 | 2 |
| 16 | 41 | 3 | 53 | 10 | 0.8 | 67 | 6.2 | 2.5 | 101 | 87 | 73 | 97 | 0.54 | 18 | 20 | 2 |
| | | Avg. | 60 | 9 | 0.68 | 57.75 | 6.575 | 2.83 | 102.75 | 77.75 | 174.75 | 117.25 | 0.56 | 38.25 | 20 | 2 |
| 6 | 31 | 4 | 50 | 7 | 0.8 | 39 | 6.8 | 3 | 101 | 81 | 152 | 97 | 0.45 | 15 | 20 | 2 |
| 10 | 35 | 4 | 45 | 9 | 0.6 | 57 | 7 | 3.2 | 102 | 179 | 214 | 73 | 0.44 | 15 | 20 | 2 |
| 11 | 36 | 4 | 52 | 10 | 0.7 | 53 | 7.3 | 3.1 | 80 | 60 | 326 | 77 | 0.59 | 22 | 20 | 2 |
| 12 | 37 | 4 | 56 | 19 | 0.6 | 62 | 6.9 | 3 | 115 | 83 | 498 | 89 | 0.54 | 37 | 20 | 2 |
| | | Avg. | 51 | 11.25 | 0.68 | 52.75 | 7 | 3.08 | 99.5 | 100.75 | 297.5 | 84 | 0.51 | 22.25 | 20 | 2 |

TABLE 6
Results after 26 days of feeding
15 Aug. 2017

| Goat | Tag | | | | | | Total | | | Creatine | Alk | | Total | Hemolytic | Lipemic | Iceteric |
|---|---|---|---|---|---|---|---|---|---|---|---|---|---|---|---|---|
| No | ID | Pen | Wt. | BUN | Creat | Glucose | Protein | Albumin | AST | Kinase | Phos | GGT | Bilirubin | Indice | Indice | Indice |
| 1 | 26 | 1 | 77.0 | 11.0 | 0.9 | 53.0 | 6.6 | 2.5 | 74.0 | 110.0 | 151.0 | 78.0 | 0.4 | 15.0 | 20.0 | 2.0 |
| 2 | 27 | 1 | 82.0 | 14.0 | 0.7 | 60.0 | 6.9 | 2.7 | 85.0 | 99.0 | 243.0 | 113.0 | 0.5 | 15.0 | 20.0 | 2.0 |
| 3 | 28 | 1 | 73.0 | 6.0 | 0.6 | 63.0 | 6.2 | 2.6 | 87.0 | 94.0 | 212.0 | 96.0 | 0.3 | 15.0 | 20.0 | 2.0 |
| 4 | 29 | 1 | 62.0 | 13.0 | 0.6 | 49.0 | 6.9 | 2.6 | 84.0 | 111.0 | 261.0 | 105.0 | 0.5 | 24.0 | 20.0 | 2.0 |
| | | Avg. | 73.5 | 11.0 | 0.7 | 56.3 | 6.7 | 2.6 | 82.5 | 103.5 | 216.8 | 98.0 | 0.4 | 17.3 | 20.0 | 2.0 |
| 7 | 32 | 2 | 56.0 | 6.0 | 0.5 | 58.0 | 6.5 | 2.1 | 78.0 | 72.0 | 133.0 | 81.0 | 0.4 | 15.0 | 20.0 | 2.0 |
| 8 | 33 | 2 | 57.0 | 5.0 | 0.5 | 48.0 | 6.9 | 2.5 | 85.0 | 178.0 | 174.0 | 95.0 | 0.7 | 59.0 | 20.0 | 2.0 |
| 9 | 34 | 2 | 52.0 | 15.0 | 0.7 | 59.0 | 5.4 | 2.0 | 72.0 | 120.0 | 123.0 | 76.0 | 0.6 | 27.0 | 20.0 | 2.0 |
| 15 | 40 | 2 | 54.0 | 16.0 | 0.7 | 51.0 | 6.3 | 2.3 | 74.0 | 178.0 | 142.0 | 94.0 | 0.4 | 23.0 | 20.0 | 2.0 |
| | | Avg. | 54.8 | 10.5 | 0.6 | 54.0 | 6.3 | 2.2 | 77.3 | 137.0 | 143.0 | 86.5 | 0.5 | 31.0 | 20.0 | 2.0 |
| 5 | 30 | 3 | 78.0 | 13.0 | 0.5 | 61.0 | 7.4 | 3.2 | 100.0 | 98.0 | 398.0 | 103.0 | 0.4 | 16.0 | 20.0 | 2.0 |
| 13 | 38 | 3 | 72.0 | 14.0 | 0.5 | 59.0 | 7.5 | 3.1 | 96.0 | 193.0 | 301.0 | 107.0 | 0.6 | 21.0 | 20.0 | 2.0 |
| 14 | 39 | 3 | 85.0 | 14.0 | 0.5 | 72.0 | 6.9 | 3.2 | 91.0 | 77.0 | 268.0 | 123.0 | 0.4 | 31.0 | 20.0 | 2.0 |
| 16 | 41 | 3 | 77.0 | 18.0 | 0.6 | 48.0 | 6.6 | 2.8 | 105.0 | 184.0 | 262.0 | 97.0 | 0.5 | 26.0 | 20.0 | 2.0 |
| | | Avg. | 78.0 | 14.8 | 0.5 | 60.0 | 7.1 | 3.1 | 98.0 | 138.0 | 307.3 | 107.5 | 0.5 | 23.5 | 20.0 | 2.0 |
| 6 | 31 | 4 | 61.0 | 15.0 | 0.6 | 57.0 | 6.9 | 3.0 | 96.0 | 98.0 | 326.0 | 101.0 | 0.4 | 15.0 | 20.0 | 2.0 |
| 10 | 35 | 4 | 63.0 | 12.0 | 0.5 | 68.0 | 6.9 | 3.0 | 103.0 | 93.0 | 442.0 | 76.0 | 0.4 | 15.0 | 20.0 | 2.0 |
| 11 | 36 | 4 | 63.0 | 15.0 | 0.5 | 47.0 | 6.9 | 3.0 | 117.0 | 90.0 | 433.0 | 75.0 | 0.6 | 37.0 | 20.0 | 2.0 |
| 12 | 37 | 4 | 72.0 | 16.0 | 0.5 | 68.0 | 6.9 | 3.0 | 90.0 | 106.0 | 489.0 | 80.0 | 0.5 | 19.0 | 20.0 | 2.0 |
| | | Avg. | 64.8 | 14.5 | 0.5 | 60.0 | 6.9 | 3.0 | 101.5 | 96.8 | 422.5 | 83.0 | 0.5 | 21.5 | 20.0 | 2.0 |

As shown in Tables 5 and 6, those pens with additive gained additional weight over those without the additive. For example, Pen 1, gained an average of 9.5 pounds, whereas Pen 2 averaged a gain of 3.5 pound. Similarly, Pen 3 gained an average 18 pounds, whereas Pen 4 gained 13.8 pounds. Unexpectedly, the pens receiving the additive saw an increase in total protein and albumin in the blood stream. Such an increase contributes to overall health and increased weight gain. Other advantages to health and well-being of the animal can be seen from the data. During the course of the trials, it was also determined that less excrement was being produced by the goats fed with the additive. Without seeking to be limited to a particular theory of invention, it is believed that goats receiving the additive had increased digestive and nutrient efficiency, thus resulting in a reduced amount of excrement.

The present disclosure is not to be limited to the particular embodiments described herein. The following claims set forth a number of the embodiments of the present disclosure with greater particularity.

What is claimed is:

1. A livestock feed additive comprising calcium, phosphorus, sodium chloride, potassium, sulfur, magnesium, manganese, zinc, iron, copper, selenium, vitamin A, Vitamin E, and mendione.

2. A method of improving livestock health and performance comprising providing to said livestock a dietary supplement substantially according to the formulation of claim 1.

3. The method of claim 2 wherein the livestock health and performance to be improved relates to one or more of fertility, hair growth, eye health, feed intake, feed efficiency, productive milk output, milk quality indices, and/or meat production.

4. A dietary supplement according to the formulation of claim 1, wherein said supplement is in the form of a powder, liquid, or solid.

5. The dietary supplement of claim 4, wherein said supplement is provided as, or integrated into, a feed block.

6. A livestock animal with improved health and performance produced by any of the methods of claim 5.

7. A livestock feed additive comprising sodium bentonite, sodium sulfate, dried Aspergillus oryzae fermentation product, ferrous sulfate, calcium carbonate, manganese proteinate, manganese sulfate, molasses products, vegetable oil, lecithin, high fructose corn syrup, polysorbate 60, glycerin, sulfuric acid, xanthan gum, natural and artificial flavor, zinc proteinate, selenium yeast, zinc sulfate, yeast culture, vitamin A supplement, vitamin D-3 supplement, vitamin E supplement, choline chloride, niacin, calcium pantothenate, riboflavin, vitamin B-12 supplement, thiamine hydrochloride, copper sulfate, copper proteinate, Lactobacillus acidophilus, Lactobacillus brevis, Lactobacillus plantarum, Enterococcus faecium, and Pediococcus acidlacticii.

8. A method of improving livestock health and performance comprising providing to said livestock a dietary supplement substantially according to the formulation of claim 7.

9. The method of claim 8 wherein the livestock health and performance to be improved relates to one or more of fertility, hair growth, eye health, feed intake, feed efficiency, productive milk output, milk quality indices, and/or meat production.

10. A dietary supplement according to the formulation of claim 7, wherein said supplement is in the form of a powder, liquid, or solid.

11. The dietary supplement of claim 10, wherein said supplement is provided as, or integrated into, a feed block.

12. A livestock animal with improved health and performance produced by any of the methods of claim 11.

13. A livestock drench composition comprising crude protein, crude fat, fiber, copper, manganese, zinc, vitamin E, phosphorus, sodium chloride, magnesium, potassium, iron, iodine, and sulfur.

14. A method of improving livestock health and performance comprising providing to said livestock a dietary supplement substantially according to the formulation of claim 13.

15. The method of claim 14, wherein the livestock health and performance to be improved relates to one or more of fertility, hair growth, eye health, feed intake, feed efficiency, productive milk output, milk quality indices, and/or meat production.

16. A dietary supplement according to the formulation of claim 15, wherein said supplement is in the form of a powder, liquid, or solid.

17. The dietary supplement of claim 16, wherein said supplement is provided as, or integrated into, a feed block.

18. A livestock animal with improved health and performance produced by any of the methods of claim 17.
